(19) United States
(12) Patent Application Publication
(10) Pub. No.: US 2023/0365929 A1
(43) Pub. Date: Nov. 16, 2023

(54) METHOD OF DIFFERENTIATION OF PLURIPOTENT STEM CELLS TO RETINAL PIGMENT EPITHELIUM CELLS

(71) Applicant: R.P. SCHERER TECHNOLOGIES, LLC, Carson City, NV (US)

(72) Inventors: Boris GREBER, Düsseldorf (DE); Melanie HUEHNE, Niederkassel (DE)

(21) Appl. No.: 18/195,849

(22) Filed: May 10, 2023

Related U.S. Application Data
(60) Provisional application No. 63/340,736, filed on May 11, 2022.

(51) Int. Cl. C12N 5/079; A61P 27/02

(52) U.S. Cl. CPC C12N 5/0621; A61P 27/02; C12N 2506/45; C12N 2501/15; C12N 2501/155; C12N 2501/119; C12N 2501/16; C12N 2533/52

(57) ABSTRACT

The invention provides a method of generating retinal pigmented epithelial (RPE) cells from pluripotent stem cells (PSCs), and methods of use of the cells. The disclosure provides a method of generating RPE cells by differentiation of PSCs and includes the treatment of PSCs with one or more of a transforming growth factor β/SMAD2/SMAD3 pathway signaling inhibitor, a bone morphogenetic protein/SMAD1/SMAD5/SMAD8 pathway signaling inhibitor or a fibroblast growth factor/ERK pathway signaling inhibitor, and a TGFβ family protein. Use of such RPE cells include methods of treating macular degeneration by administering the RPE cells to a subject in need thereof.

### CROSS-REFERENCE TO RELATED APPLICATIONS

This application claims benefit of priority under 35 U.S.C. § 119(e) of U.S. Provisional Application No. 63/340,736, filed May 11, 2022. The disclosure of the prior application is considered part of and is herein incorporated by reference in the disclosure of this application in its entirety.

### BACKGROUND OF THE INVENTION

### Field of the Invention

The present invention relates generally to retinal pigment epithelium (RPE) cells, and more specifically to methods of generating RPE cells from pluripotent stem cells (PSCs).

### Background Information

Retinal pigment epithelium (RPE) forms a single cell-layered sheet of cells underlying the photoreceptors of the eye. It is essential for the function and survival of these retinal photoreceptors and, therefore, for proper vision. Amongst other functions, the RPE cells contribute to the renewal of photoreceptor outer segments (POS) by phagocytosis involving MERTK. Through the formation of tight junctions, the RPE layer also forms the brain-eye barrier separating the inner eye from the blood stream. Degenerating or otherwise damaged RPE gives rise to macular degeneration which may be potentially treated by the in-situ transplantation of RPE cells generated from human pluripotent stem cells (hPSCs), including induced pluripotent stem cells (iPSCs), or embryonic stem cells (ESCs).

Different approaches have been devised for promoting RPE differentiation from PSCs, often using basal media containing various molecules. For instance, RPE induction was described using a complex cocktail of small molecules and growth factors including Noggin, Dkk1, IGF1, bFGF, Activin A, nicotinamide, SU5402, and others, in the presence of bovine serum albumin. Some studies used nicotinamide and Activin A or combinations of casein kinase/TGFβ/SMAD2/3 inhibitors, at limited efficiency to drive RPE induction (Idelson et al., 2009; Osakada et al., 2009). Other combination and sequence of adding the differentiation-driving molecules on an animal-derived extracellular matrix were also described, using for example bFGF, SB431542, retinoic acid, Shh, and Noggin (Zahabi et al., 2012). Often, the individual effects exerted by such molecules are not established and those “cocktails” are not optimized with regards to exact composition, factor concentrations, timing of administration, and so forth.

RPE cells may also, slowly (within several weeks) and at reduced efficiency, be obtained through spontaneous differentiation along the neural pathway (Plaza Reyes et al., 2016). Indeed, it was demonstrated that human embryonic stem cells (hESCs) could be differentiated into RPE cells by culturing them in a xeno-free and defined Nutri Stem™ hESC XF medium in the absence of basic fibroblast growth factor (bFGF) using recombinant human laminin (rhLN-521)-based matrix. In such conditions, hESC-RPE differentiated cells having a reduction of pluripotency-associated transcripts OCT3/4 and NANOG, together with robust expression of neuroectoderm transcripts sex-determining region Y-box 9 protein (SOX9) and paired box 6 (PAX6) were obtained. This culture robustly supports the formation of pigmented structures resembling optical vesicles (OVs) at as early as 3 weeks of differentiation, with homogeneous pigmentation obtained by week 9.

Another alternative approach uses chetonin, an inhibitor of hypoxia-induced factor (HIF) signaling, as a driver of RPE fate (Maruotti et al., 2015; Sharma et al., 2019).

The existing methods yield cells population that are not pure, rely on the use of animal-derived molecules (which is an issue for clinical compliance and reduced immunogenicity) and may take weeks to obtain. There is still a need in the art to develop methods for RPE differentiation that are efficient and yield pure cell cultures in a short period of time using convenient culture conditions.

### SUMMARY OF THE INVENTION

The present invention is based on the seminal discovery that a combination of a neuroectodermal induction cocktail transforming growth factor beta (TGF-beta) family pathway inhibitory molecules, and/or a fibroblast growth factor (FGF)/ERK pathway signaling inhibitory molecule with Activin A treatment yields induction of RPE cells from PSCs when used in a defined temporal sequence and in adherent cell culture conditions.

In one embodiment, the present invention provides a method of generating retinal pigment epithelium (RPE) cells including: (a) contacting a culture of pluripotent stem cells (PSCs) with a mixture of agents including: (i) one or more of a transforming growth factor TGFβ (TGFβ)/SMAD2/SMAD3 pathway signaling inhibitor, a bone morphogenetic protein (BMP)/SMAD1/SMAD5/SMAD8 pathway signaling inhibitor or a fibroblast growth factor (FGF)/ERK pathway signaling inhibitor; and (ii) optionally, a TGFβ family protein; followed by (b) culturing the cells of (a) with the TGFβ family protein in the absence of the mixture of the agents of (a)(i).

In one aspect, the culture of PSCs is an adherent monolayer of cells. In one aspect, the monolayer of cells is grown in a two-dimensional culture system, e.g., a petri dish. In one aspect, the TGFβ/SMAD2/SMAD3 pathway signaling inhibitor is selected from the group consisting of SB431542, LY3200882, TP0427736 HCl, RepSox, SB525334, GW788388, BIBF-0775, SD-208, galunisertib, vactosertib, A-83-01, LY2109761, SB505124, LY364947 and LDN-212854. In another aspect, the BMP/SMAD1/SMAD5/SMAD8 pathway signaling inhibitor is selected from the group consisting of dorsomorphin, PPM1A and LDN-193189. In an additional aspect, the FGF/ERK pathway signaling inhibitor is selected from the group consisting of PD0325901, PD173074, SU431542, PD161570, PD98059, PD184352, PD198306 and PD334581. In one aspect, the TGFβ protein superfamily member is selected from the group consisting of Activin A, TGFβ1, TGFβ2 and TGFβ3.

In one aspect, the mixture of (a) includes about 0.1-10 μM TGFβ/SMAD2/SMAD3 pathways signaling inhibitor, about 0.1-1 μM BMP/SMAD1/SMAD5/SMAD8 pathway signaling inhibitor, and/or about 0.5-5 μM FGF/ERK pathway signaling inhibitor; and at least about 0.1 ng/ml of a TGFβ family protein. In various aspects, the mixture includes about 1-5 μM SB431542, about 0.25 μM dorsomorphin, about 0.5-1.5 μM PD0325901, and about 25 ng/ml Activin A. In one aspect, contacting PSCs with the TGFβ family protein comprises contacting the PSCs with at least about 0.1 ng/ml TGFβ family protein. In one aspect, contacting PSCs with the TGFβ family protein comprises contacting the PSCs with at least about 5 ng/ml Activin A. In another aspect, contacting PSCs with the TGFβ family protein includes contacting PSCs with about 25 ng/ml Activin A. In one aspect, contacting PSCs with the mixture is for about 2-7 days. In another aspect, contacting PSCs with the TGFβ family protein (e.g., Activin A) is for about 4 weeks.

In some aspects, the monolayer is cultured on a surface comprising a laminin coating. In various aspects, the PSCs are human PSCs (hPSCs), such as human induced pluripotent stem cells (hiPSCs) or human embryonic stems cells (hESCs). In one aspect, contacting PSCs with the mixture is in the absence of a hypoxia inducible factor (HIF) pathway modulator. In another aspect, contacting PSCs with the mixture is in the absence of nicotinamide. In an additional aspect, the PSCs are cultured under conditions that are not hypoxic conditions, e.g., under normoxic conditions. In a further aspect, the hPSCs are cultured in a system that is not in a three-dimensional culture system (e.g., a spinner flask).

In another embodiment, the invention provides a method of inducing retinal pigment epithelium (RPE) cell differentiation from pluripotent stem cells (PSCs) including: (a) culturing PSCs in conditions that allow growth as an adherent monolayer in a two-dimensional culture system; (b) contacting the PSCs with a mixture of agents including one or more of a TGFβ/SMAD2/SMAD3 pathway signaling inhibitor, a BMP/SMAD1/SMAD5/SMAD8 pathway signaling inhibitor, or a FGF/ERK pathway signaling inhibitor and optionally a TGFβ superfamily protein for about 2-7 days; and (c) subsequently culturing the cells of (b) with the TGFβ family protein for about 4 additional weeks.

In one aspect, RPE cell differentiation is direct RPE cell differentiation. In another aspect, differentiated RPE cells have an increased expression of PMEL17, MITF, OTX2, BEST1, RPE65, RLBP1, CLDN19, ATP1B1, NC1, ZO1 and/or TYR as compared to PSCs. In one aspect, differentiated RPE cells do not express ECAT11, OCT4, NANOG, SOX2, mir302HT and/or LIN28.

In an additional embodiment, the invention provides a method of treating macular degeneration in a subject including administering to the subject differentiated retinal pigment epithelium (RPE) cells, wherein differentiated RPE cells are obtained by one of the methods described herein.

In one aspect, administering differentiated RPE cells increases photoreceptor function and/or survival. In some aspects, increasing photoreceptor function comprises increasing renewal of photoreceptor outer segment, increasing phagocytosis involving MERTK and/or increasing, restoring and/or creating cell/cell tight junctions. In various aspects, restoring and/or creating cell/cell tight junctions improves or restores brain-eye barrier. In one aspect, administering differentiated RPE cells comprises injecting RPE cells in situ. In another aspect, hPSCs are autologous hPSCs or allogenic hPSCs.

In a further embodiment, the invention provides a kit comprising: (a) a neuroectodermal induction cocktail including: a TGFβ/SMAD2/SMAD3 pathway signaling inhibitor, a BMP/SMAD1/SMAD5/SMAD8 pathway signaling inhibitor, and/or an FGF/ERK pathway signaling inhibitor; (b) a TGFβ family protein; and (c) instructions for inducing pluripotent stem cells (PSCs) differentiation into retinal pigment epithelium (RPE) cells.

In one aspect, the kit further includes a laminin-coated surface.

### BRIEF DESCRIPTION OF THE DRAWINGS

FIG. 1 is an illustrative schematic representation of a method of the invention.

FIGS. 2A-2C illustrate RPE cell differentiation protocol performance as compared to a reference protocol. FIG. 2A is a graph bar showing the expression of RPE-specific markers as obtained by RTqPCR in cells differentiated using a reference protocol or the protocol of the present invention. FIG. 2B is a photograph comparing pigmentation of the cells obtained by the two protocols. FIG. 2C is a graph bar showing the expression of RPE-specific markers as obtained by RTqPCR in non-treated cells, cells treated with Activin A only and cells differentiated using the protocol of the present invention.

FIG. 3 is a bar graph illustrating the expression of RPE-specific markers as obtained by RTqPCR in cells differentiated using a PSD cocktail, no PD, no SB or no DM. PSD cocktail: TGFβ/SMAD2/SMAD3 inhibitor+BMP/SMAD1/SMAD5/SMAD8 inhibitor+FGF/ERK inhibitor; no PD: TGFβ/SMAD2/SMAD3 inhibitor+BMP/SMAD1/SMAD5/SMAD8 inhibitor; no SB: BMP/SMAD1/SMAD5/SMAD8 inhibitor+FGF/ERK inhibitor; no DM: TGFβ/SMAD2/SMAD3 inhibitor+FGF/ERK inhibitor.

FIGS. 4A-4B are graphs illustrating RPE-specific markers expression in cells differentiated with PD+A or PSD+A. FIG. 4A shows graphs illustrating RPE-specific markers expression as measured by FACS. FIG. 4B is a graph bar illustrating RPE-specific markers expression as measured by RTqPCR. PSD+A: TGFβ/SMAD2/SMAD3 inhibitor+BMP/SMAD1/SMAD5/SMAD8 inhibitor+FGF/ERK inhibitor+Activin A; PD+A: FGF/ERK inhibitor+BMP/SMAD1/SMAD5/SMAD8 inhibitor+Activin A.

FIG. 5 is a graph illustrating OTX2, MITF and PMEL expression in cells during a 7-days PSD+A RPE differentiation.

FIG. 6 is a graph bar illustrating RPE-specific markers expression in cells differentiated with PSD then A or PSD+A.

FIG. 7 is a graph illustrating the titration of Activin A treatment.

FIGS. 8A-8C are graphs illustrating the expression of early, mid/late, late REP markers and maturation markers in cells differentiated using a PSD+A protocol, and as measured by RTqPCR. FIG. 8A is a graph illustrating early RPE markers OTX2, MITF, PMEL and ATP1B1 expression. FIG. 8B is a graph illustrating mid/late RPE markers TYR and CLDN19 expression. FIG. 8C is a graph illustrating late RPE/maturation markers RPE65, RLBP1 and BEST1 expression.

FIGS. 9A-9D are photographs illustrating phase contrast and immunofluorescently labeled RPE cells obtained by a PSD+A protocol. FIG. 9A is a photograph illustrating RPE cells in phase contrast. FIG. 9B is a photograph illustrating BEST1 and ZO1 protein expression in RPE cells. FIG. 9C is a photograph illustrating MITF1 and CRALBP protein expression in RPE cells. FIG. 9D is a photograph illustrating CRALBP and ZO1 protein expression in RPE cells.

FIGS. 10A-10B are photographs illustrating electron microscopy analysis of iPSC-derived RPE cells generated using a PSD+A protocol. FIG. 10A is a photograph illustrating transmission electron microscopy of the cells. FIG. 10B is a photograph illustrating scanning electron microscopy of the cells.

FIG. 11 is a graph bar illustrating the expression of iPSC markers in iPSC derived RPE cells as measured by RTqPCR.

### DETAILED DESCRIPTION OF THE INVENTION

The present invention is based on the seminal discovery that a combination of a neuroectodermal induction cocktail including one or more of TGFβ family pathway inhibitory molecules and/or FGF/ERK pathway signaling inhibitory molecules with a TGFβ family protein yields induction of RPE cells from PSCs when used in a defined temporal sequence and in adherent cell culture conditions.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of generating retinal pigment epithelium (RPE) cells including: (a) contacting a culture of pluripotent stem cells (PSCs) with a mixture of agents including: (i) one or more of a transforming growth factor TGFβ (TGFβ)/SMAD2/SMAD3 pathway signaling inhibitor, a bone morphogenetic protein (BMP)/SMAD1/SMAD5/SMAD8 pathway signaling inhibitor, or a fibroblast growth factor (FGF)/ERK pathway signaling inhibitor; and (ii) optionally, a TGFβ family protein; and (b) subsequently culturing the cells of (a) with the TGFβ family protein in the absence of the mixture of the agents of (a)(i).

The pigmented layer of retina or retinal pigment epithelium (RPE) is the pigmented cell layer just outside the neurosensory retina that nourishes retinal visual cells and is firmly attached to the underlying choroid and overlying retinal visual cells. The RPE is composed of a single layer of hexagonal cells (RPE cells) that are densely packed with pigment granules. When viewed from the outer surface, these cells are smooth and hexagonal in shape. When seen in section, each cell consists of an outer non-pigmented part containing a large oval nucleus and an inner pigmented portion which extends as a series of straight thread-like processes between the rods, this being especially the case when the eye is exposed to light.

The RPE has several functions including light absorption, epithelial transport, spatial ion buffering, visual cycle, phagocytosis, secretion and immune modulation. RPE cells are responsible for absorbing scattered light. This role is very important for two main reasons, first, to improve the quality of the optical system, second, light is radiation, and it is concentrated by a lens onto the cells of the macula, resulting in a strong concentration of photo-oxidative energy. Melanosomes absorb the scattered light and thus diminish the photooxidative stress. The high perfusion of retina brings a high oxygen tension environment. The combination of light and oxygen brings oxidative stress, and RPE has many mechanisms to cope with it. RPE cells compose the outer blood-retinal barrier, the epithelium has tight junctions between the lateral surfaces and implies an isolation of the inner retina from the systemic influences. This is important for the immune privilege (not only as barrier, but with signaling process as well) of eyes, a highly selective transport of substances for a tightly controlled environment. RPE supply nutrients to photoreceptors, control ion homeostasis and eliminate water and metabolites. The visual cycle fulfills an essential task of maintaining visual function and needs therefore to be adapted to different visual needs such as vision in darkness or lightness. Photoreceptor outer segment (POS) membranes are exposed to constant photo-oxidative stress, and they go through constant destruction by it. They are constantly renewed by shedding their end, which RPE then phagocytose and digest. The RPE is an epithelium which closely interacts with photoreceptors on one side but must also be able to interact with cells on the blood side of the epithelium, such as endothelial cells or cells of the immune system. In order to communicate with the neighboring tissues, the RPE is able to secrete a large variety of factors and signaling molecules. It secretes ATP, fas-ligand (fas-L), fibroblast growth factors (FGF-1, FGF-2, and FGF-5), transforming growth factor-β (TGF-β), insulin-like growth factor-1 (IGF-1), ciliary neurotrophic factor (CNTF), platelet-derived growth factor (PDGF), vascular endothelial growth factor (VEGF), lens epithelium-derived growth factor (LEDGF), members of the interleukin family, tissue inhibitor of matrix metalloproteinase (TIMP) and pigment epithelium-derived factor (PEDF). Many of these signaling molecules have important physio-pathologic roles. The inner eye represents an immune privileged space which is disconnected from the immune system of the blood stream. The immune privilege is supported by the RPE in two ways. First, it represents a mechanical and tight barrier which separates the inner space of the eye from the blood stream. Second, the RPE is able to communicate with the immune system in order to silence immune reaction in the healthy eye or, on the other hand, to activate the immune system in the case of disease.

Dysfunction of the RPE is associated with RPE diseases that can be referred to as retinal degenerative diseases. Retinal degenerative diseases can result from oxidative stress and inflammation, apoptosis and autophagy, and/or defect in cell polarity and interactions.

The cornea has a transparent structure and the RPE is exposed to light for long periods of time, has a rich oxygen supply, and consequently large amounts of reactive oxygen are easily generated. In addition, increased systemic glucose levels, such as in diabetics, can facilitate excessive ROS accumulation. In degenerative retinopathy, antioxidant levels decrease in cells; that is, the capacity of RPE cells to remove ROS variably reduces, resulting in a large accumulation of POS. Oxidative stress and inflammation cause RPE cell damage, which in turn causes retinal dysfunction and even blindness. Diseased RPE exhibits increased apoptosis, autophagy, and endoplasmic reticulum stress levels than normal cells. RPE cell death via apoptosis and endoplasmic reticulum stress has been observed in age-related macular degeneration (AMD) and other retinal degenerative diseases. The polarity and cell junctions of the RPE play critical roles in the blood-retinal barrier, maintaining the stability of the internal photoreceptor microenvironment and supporting the choroidal system. Disrupted cell polarity and cell junctions significantly increase the risk of retinal degenerative disease. RPE polarity and cell junction stability are related to the unique basal and apical structures of the retina, which affect phagocytosis and material exchange. Abnormalities in RPE polarity, barrier destruction, and retinal stability may contribute to the pathogenesis of blinding retinal diseases.

There is no curative treatment for retinal pigment epithelial diseases. Thanks to many years of research into retinal diseases, many genes and signal transduction pathways have been identified as potential targets for gene therapy or other therapeutics. Cell therapy, using stem cell derived RPE and photoreceptors have restored vision in pre-clinical models of human retinal degenerative diseases. Stem cell transplantation is therefore an effective approach to treat RPE diseases.

The transforming growth factor beta (TGF-beta) superfamily includes TGF-beta proteins, bone morphogenetic proteins (BMPs), growth differentiation factors (GDFs), glial-derived neurotrophic factors (GDNFs), Activins, Inhibins, Nodal, Lefty, and Mülllerian inhibiting substance (MIS). Ligands of the TGF-beta superfamily form dimers that bind to heterodimeric receptor complexes consisting of type I and type II receptor subunits with serine/threonine kinase domains. Following ligand binding, the type II receptor phosphorylates and activates the type I receptor, initiating a Smad-dependent signaling cascade that induces or represses transcriptional activity. During development, members of the TGF-beta family are thought to be required for dorso-ventral patterning, mesoderm induction and patterning, limb bud formation, bone and cartilage formation, neuron differentiation, and the development of a variety of different tissues and organs.

A “pathway signaling inhibitor” as used herein refers to any molecule that is capable of inhibiting a signaling pathway of interest. A signaling pathway is a series of chemical reactions in which a group of molecules in a cell work together to control a cell function, such as cell differentiation. A cell receives signals from its environment when a molecule, such as a hormone or growth factor, binds to a specific protein receptor on or in the cell. After the first molecule in the pathway receives a signal, it activates another molecule. This process is repeated through the entire signaling pathway until the last molecule is activated and the cell function is carried out. Abnormal activation of signaling pathways, or inhibition of a signaling pathway may lead to diseases, or, in the case of pluripotent cells to alteration of the pluripotent state, and therefore to differentiation. The term “molecule” includes, but is not limited to, small molecules (including small molecules that do not have optimal cell-permeability), lipids, nucleosides, nucleotides, nucleic acids, polynucleotides, oligonucleotides, antibodies, toxins, negatively charged polymers and other polymers, for example proteins, peptides, hormones, carbohydrates, or polyamines. Non-limiting examples of polynucleotides include short interfering nucleic acid (siNA), antisense, enzymatic nucleic acid molecules, 2′,5′-oligoadenylate, triplex forming oligonucleotides, aptamers, and decoys. Biologically active molecules include antibodies (e.g., monoclonal, chimeric, humanized etc.), cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, allozymes, aptamers, decoys and analogs thereof, and small nucleic acid molecules, such as short interfering nucleic acid (siNA), short interfering RNA (siRNA), double-stranded RNA (dsRNA), micro-RNA (miRNA), antagomirs, and short hairpin RNA (shRNA) molecules.

A “TGFβ/SMAD2/SMAD3 pathway signaling inhibitor”, a BMP/SMAD1/SMAD5/SMAD8 pathway signaling inhibitor”, or an “FGF/ERK pathway signaling inhibitor” as used herein refer to any molecule capable of inhibiting the TGFβ/SMAD2/SMAD3, the BMP/SMAD1/SMAD5/SMAD8 or the FGF/ERK signaling pathway, respectively. Signaling pathway inhibition is the opposite of signaling pathway upregulation. In this process, small molecules called “signal transduction inhibitors” or “pathway signaling inhibitors” block the communication between different molecules of the pathway and interrupt the molecular signaling cascade.

A “TGFβ superfamily protein” or “TGFβ family protein”, as used herein refers to any protein or peptide of the transforming growth factor beta (TGF-β) superfamily, a large group of structurally related cell regulatory proteins that was named after its first member, TGFβ1. TGFβ proteins interact with TGF-beta receptors. Many proteins have since been described as members of the TGFβ family in a variety of species, including invertebrates as well as vertebrates and categorized into 23 distinct gene types that fall into four major subfamilies: the TGF-β subfamily, including TGFβ1, TGFβ2 and TGFβ3; the bone morphogenetic proteins and the growth differentiation factors; the activin and inhibin subfamilies; and the left-right determination factors. Non-limiting examples of TGFβ family protein for use in the present methods include Activin A, TGFβ1, TGFβ2 and TGFβ3.

The methods described herein describe cellular culture conditions in which human pluripotent stem cells are grown, that yield the generation of RPE cells.

Stem cells are undifferentiated cells that have the ability to self-renew indefinitely and to remain in said undifferentiated state. As opposed to embryonic stem cells which can only be isolated from the inner mass of a blastocyst, there are three known accessible sources of adult stem cells: the bone marrow which requires the drilling of a bone, the adipose tissue which is accessible by liposuction, and the blood, from which the cells can be extracted among other cells. The term “pluripotent stem cells”, as used herein refers to cells that are capable of generating all the cell types of an organism, i.e., cells derived from any of the three germ layers. On the other hand, multipotent stem cells can differentiate into several cell type, but only those of a closely related family of cells, generally the cell types of the organ from which they originate. Most adult stem cells are multipotent but small amounts of pluripotent adult stem cells can be retrieved from umbilical cord or other tissues. The sources of cells used for retinal cell therapy include stem cells such as embryonic stem cells (ESCs), adult stem cells, and induced pluripotent stem cells (iPSCs). Currently, ESCs, and iPSCs are mainly used for differentiation into RPE.

In some aspects, the PSCs used in the methods described herein are human, and in some instances the human PSCs are induced pluripotent stem cells (hiPSCs) or human embryonic stems cells (hESCs).

By “generating” RPE cells, it is meant that the present methods provide physical and chemical culture conditions that have been optimized to induce the differentiation of PSCs into RPE cells. The differentiation method described herein yields RPE cell population that are enriched for RPE cells. For example, greater than 80%, greater than 85%, greater than 90%, greater than 95%, 96%, 97%, 98% or 99% RPE cells are obtained in shorter times and using more convenient cultures conditions than the methods available in the art.

Physical culture conditions include but are not limited to the culture environment of the cell (e.g., adherent versus suspension culture, or in two-dimensional versus in three-dimensional culture systems), the pH of the culture media, the gas concentration in the incubator (e.g., CO₂ concentration, O₂ concentration), and the temperature.

There are two basic systems for growing cells in culture, as monolayers on an artificial substrate (i.e., adherent culture) or free-floating in the culture medium (suspension culture). The majority of the cells derived from vertebrates, with the exception of hematopoietic cell lines and a few others, are anchorage-dependent and have to be cultured on a suitable substrate that is specifically treated to allow cell adhesion and spreading (i.e., tissue-culture treated). However, many cell lines can also be adapted for suspension culture.

In one aspect, the culture of PSCs is an adherent monolayer of cells. In another aspect, the monolayer of cells is grown in a two-dimensional culture system. In a further aspect, the PSCs are cultured in a system that is not in a three-dimensional culture system.

In addition to the treatment of the tissue-culture surface, cells can require to be grown on coated surfaces, to enhance or improve their adhesion and/or spreading (i.e., using a coating). “Coating” as an additional surface treatment stands for all additional modifications made to increase cell adhesion in addition to the standard plasma or corona treatment which is performed on all cell culture plastic by manufacturer. Usually, coating is done with proteins or peptides. Various proteins can be used to coat tissue-culture treated dishes, including poly-L-Lysine, poly-D-Lysine, poly-Ornithine, gelatin, collagen I, IV, fibronectin, laminin, vitronectin, osteopontin, fibronectin domains, Matrigel™ (several components of the extracellular matrix with bound growth factors etc.), collagen gels, alginate gels, and lactate gels.

In one aspect, the monolayer is cultured on a surface comprising a laminin coating.

Physical culture conditions include the gas concentration in the incubator. Incubation of cell cultures is typically performed in normal atmosphere with 15-22% oxygen and 5% CO₂ for expansion and seeding. In various aspects, the PSCs are grown in a humidified atmosphere including about 5% CO₂ concentration, and normoxic conditions (non-hypoxic O₂ concentration). While hypoxic culture conditions are thought to support stem cell performance in general, in the present methods, the PSCs are cultured under conditions that are not hypoxic conditions. As used herein, “normoxic” conditions refer to culture conditions including atmospheric O₂ concentration (e.g., about 15-25% O₂ concentration). As used herein, hypoxic conditions are characterized by a lower oxygen concentration as compared to the oxygen concentration of ambient air (approximately 15%-25% oxygen). In one aspect, hypoxic conditions are characterized by an oxygen concentration less than about 10%. In another aspect hypoxic conditions are characterized by an oxygen concentration of about 0.1% to 10%, 1% to 10%, 1% to 9%, 1% to 8%, 1% to 7%, 1% to 6%, 1% to 5%, 1% to 4%, 1% to 3%, or 1% to 2%. For example, hypoxic conditions include culture conditions with 0.1, 0.2, 0.3, 0.4, 0.5, 0.6, 0.7, 0.8, 0.9, 1, 2, 3, 4, 5, 6, 7, 8, 9 or 10% O₂ concentration, or any intermediate value.

Chemical culture conditions include but are not limited to the agents or molecules that are added to the culture medium to achieve the desired effects sought after (i.e., differentiation of PSCs into RPE cells). The terms “agent” and “molecule” are used interchangeably and include, but are not limited to, small molecules (including small molecules that do not have optimal cell-permeability), lipids, nucleosides, nucleotides, nucleic acids, polynucleotides, oligonucleotides, antibodies, toxins, negatively charged polymers and other polymers, for example proteins, peptides, hormones, carbohydrates, or polyamines.

In various aspects, the chemical culture conditions of the presently described methods include a mixture of agents including one or more of a TGFβ/SMAD2/SMAD3 pathway signaling inhibitor, a BMP/SMAD1/SMAD5/SMAD8 pathway signaling inhibitor, or an FGF/ERK pathway signaling inhibitor; and optionally a TGFβ family protein.

For example, the mixture of agents includes a TGFβ/SMAD2/SMAD3 pathway signaling inhibitor, a BMP/SMAD1/SMAD5/SMAD8 pathway signaling inhibitor and a TGFβ family protein. In another example, the mixture of agents includes a TGFβ/SMAD2/SMAD3 pathway signaling inhibitor, a BMP/SMAD1/SMAD5/SMAD8 pathway signaling inhibitor, an FGF/ERK pathway signaling inhibitor and a TGFβ family protein. In an additional example, the mixture of agents includes a BMP/SMAD1/SMAD5/SMAD8 pathway signaling inhibitor, an FGF/ERK pathway signaling inhibitor and a TGFβ family protein. Optionally, the mixture includes an FGF/ERK pathway signaling inhibitor and a TGFβ family protein. In illustrative examples herein, the TGFβ family protein is Activin A.

TGFβ/SMAD2/SMAD3 pathway signaling inhibitors include for example, any molecule that inhibits TGFβ type I receptor (or ALK5), and its relatives ALK4 and ALK7. Non-limiting examples of TGFβ/SMAD2/SMAD3 pathway signaling inhibitor include SB431542, LY3200882, TP0427736 HCl, RepSox, SB525334, GW788388, BIBF-0775, SD-208, galunisertib, vactosertib, A-83-01, LY2109761, SB 505124, LY364947 and LDN-212854.

In one aspect, the TGFβ/SMAD2/SMAD3 pathway signaling inhibitor is SB431542.

The TGFβ/SMAD2/SMAD3 pathway signaling inhibitor is added to the PSC culture at a concentration that ranges from about 0.1 μM to 10 μM. For example, the PSC are grown in a culture media that includes about 0.1, 0.3, 0.5, 0.75, 1, 2, 3, 4, 5, 6, 7, 8 ,9, 10 μM or more. In one aspect, the mixture includes about 5 μM TGFβ/SMAD2/SMAD3 pathway signaling inhibitor. In another aspect, the mixture includes about 0.1-10 μM SB431542. In some aspects, the mixture includes about 1-5 μM SB431542. In other aspects, the mixture includes about 5 μM SB431542.

BMP/SMAD1/SMAD5/SMAD8 pathway signaling inhibitors include for example, any molecule that inhibits the BMP pathway by targeting the type I BMP receptors activin receptor-like kinase (ALK) 2, ALK3, and ALK6. Non-limiting examples of BMP/SMAD1/SMAD5/SMAD8 pathway signaling inhibitor include dorsomorphin, PPM1A and LDN-193189.

In one aspect, the BMP/SMAD1/SMAD5/SMAD8 pathway signaling inhibitor is dorsomorphin.

The BMP/SMAD1/SMAD5/SMAD8 pathway signaling inhibitor is added to the PSC culture at a concentration that ranges from about 0.1 μM to 1 μM. For example, the PSC are grown in a culture media that includes about 0.1, 0.2, 0.25, 0.3, 0.4, 0.5, 0.6, 0.7, 0.8, 0.9, 1 μM or more. In one aspect, the mixture includes about 0.25 μM BMP/SMAD1/SMAD5/SMAD8 pathway signaling inhibitor. In another aspect, the mixture includes about 0.1-10 μM dorsomorphin. In some aspects, the mixture includes about 0.25 μM dorsomorphin.

FGF/ERK pathway signaling inhibitors include for example, any molecule that inhibits the MEK1/2 signaling pathway. Non-limiting examples of FGF/ERK pathway signaling inhibitor include PD0325901, PD173074, SU431542, PD161570, PD98059, PD184352, PD198306 and PD334581. In one aspect, the FGF/ERK pathway signaling inhibitor is PD0325901.

The FGF/ERK pathway signaling inhibitor is added to the PSC culture at a concentration that ranges from about 0.1 μM to 5 μM. For example, the PSC are grown in a culture media that includes about 0.1, 0.25, 0.5, 1, 1.5, 2, 2.5, 3, 3.5, 4, 4.5, 5 μM or more. In one aspect, the mixture includes about 0.5-1.5 μM FGF/ERK pathway signaling inhibitor. In another aspect, the mixture includes about 0.1-5 μM PD0325901. In some aspects, the mixture includes about 0.5-1.5 μM PD0325901.

The TGFβ family protein is added to the PSC culture at a concentration of at least about 0.1 ng/ml. For example, the PSC are grown in a culture media that includes at least about 0.1, 0.2, 0.3, 0.4, 0.5, 0.75, 1, 2, 3, 4, 5, 10, 15, 20, 25, 30, 35, 40, 45, 50, 55, 60, 65, 70, 75, 80, 85, 90, 95, 100 ng/ml or more TGFβ family protein.

In one aspect, the TGFβ protein family member is selected from the group consisting of Activin A, TGFβ1, TGFβ2 and TGFβ3.

In some aspects, the TGFβ family protein is Activin A.

Activin A is a member of the TGFβ family of proteins produced by many cell types throughout development. It is a disulfide-linked homodimer (two beta-A chains) that binds to heteromeric complexes of a type I (Act RI-A and Act RI-B) and a type II (Act RII-A and Act RII-B) serine-threonine kinase receptor. Activins primarily signal through SMAD2/3 proteins to regulate a variety of functions, including cell proliferation, differentiation, wound healing, apoptosis, and metabolism. Activin A maintains the undifferentiated state of human embryonic stem cells and also facilitates differentiation of human embryonic stem cells into definitive endoderm. Activin A is added to the PSC culture at a concentration that ranges from about 0.1-100 ng/ml. For example, the PSC are grown in a culture media that includes about 0.1, 0.2, 0.3, 0.4, 0.5, 0.75, 1, 2, 3, 4, 5, 10, 15, 20, 25, 30, 35, 40, 45, 50, 55, 60, 65, 70, 75, 80, 85, 90, 95, 100 ng/ml or more Activin A. In one aspect, the mixture includes at least about 5 ng/ml Activin A. In some aspects, the mixture includes about 25 ng/ml Activin A.

In other aspects, the TGFβ protein family member is TGFβ1 or TGFβ3. TGFβ1 or TGFβ3 is added to the PSC culture at a concentration that ranges from about 0.1-100 ng/ml. For example, the PSC are grown in a culture media that includes about 0.1, 0.2, 0.3, 0.4, 0.5, 0.75, 1, 2, 3, 4, 5, 10, 15, 20, 25, 30, 35, 40, 45, 50, 55, 60, 65, 70, 75, 80, 85, 90, 95, 100 ng/ml or more TGFβ1 or TGFβ3.

The method described herein is further defined by the exclusion of chemical conditions for the culture of the PSCs. For example, the present method for generating RPE cells include the culture of PSCs is the in the absence of a hypoxia inducible factor (HIF) pathway modulator and in the absence of nicotinamide.

Hypoxia Inducible Factors (HIFs) are transcription factors that are activated in response to decreased oxygen availability in the cellular environment. They influence cell metabolism, cell survival and angiogenesis to maintain biological homeostasis. As used herein “HIF pathway modulator” includes any agent that inhibits HIF. Non-limiting examples of HIF inhibitor include adaptaquin, TAT-cyclo-CLLFVY, DMOG, echinomycin, FM19G11, GN 44028, IOX 2, KC7F2, LW 6, PX 12, TC-S 7009 and VH 298.

In one aspect, the mixture includes about 1-10 μM TGFβ/SMAD2/SMAD3 pathway signaling inhibitor, about 0.1-1 μM BMP/SMAD1/SMAD5/SMAD8, and/or about 0.5-5 μM FGF/ERK pathway signaling inhibitor; and at least about 5 ng/ml TGFβ family protein.

In some aspects, the mixture includes about 5 μM SB431542, about 0.25 μM dorsomorphin, and/or about 0.5-1.5 μM PD0325901, and about 25 ng/ml Activin A. In one aspect, the mixture includes about 5 μM SB431542, about 0.25 μM dorsomorphin, and about 25 ng/ml Activin A. In another aspect, the mixture includes about 0.25 μM dorsomorphin, about 0.5-1.5 μM PD0325901, and about 25 ng/ml Activin A. In yet another aspect, the mixture includes about 5 μM SB431542, about 0.25 μM dorsomorphin, about 0.5-1.5 μM PD0325901, and about 25 ng/ml Activin A.

In another aspect, contacting PSCs with the TGFβ family protein includes contacting PSCs with at least about 5 ng/ml TGFβ family protein.

The method described herein includes physical and chemical culture conditions, as well as sequential exposition of the PSCs to such physical and chemical conditions, for defined lengths of time. In various aspects, the methods described herein include the contacting of the PSCs with the mixture of agents for a given length of time, followed by the contacting of the PSCs with a TGFβ family protein (e.g., Activin A). For example, contacting PSCs with the mixture is for a period of time that ranges from about 2 to 7 days. Contacting PSCs with the mixture includes contacting PSCs with the mixture for about 2, 3, 4, 5, 6, 7 or more days. In one aspect, contacting PSCs with the mixture is for about 2-4 days. In various aspects, contacting PSCs with the mixture is for about 2 days.

Contacting PSCs with the TGFβ family protein (e.g., Activin A) is for a period of time that ranges from about 2 to 6 weeks. For example, contacting PSCs with the TGFβ family protein includes contacting PSCs with the TGFβ family protein for about 2, 3, 4, 5, 6 or more weeks. In one aspect, contacting PSCs with the TGFβ family protein is for about 4 weeks.

In another embodiment, the invention provides a method of inducing retinal pigment epithelium (RPE) cell differentiation from pluripotent stem cells (PSCs) including: (a) culturing PSCs in conditions that allow growth as an adherent monolayer in a two-dimensional culture system; (b) contacting the PSCs with a mixture of agents including one or more of a TGFβ/SMAD2/SMAD3 pathway signaling inhibitor, a BMP/SMAD1/SMAD5/SMAD8 pathway signaling inhibitor, or a FGF/ERK pathway signaling inhibitor; and optionally a TGFβ family protein for about 2-7 days; and (c) subsequently culturing the cells of (b) with the TGFβ family protein for about 4 additional weeks.

Cellular differentiation is the process in which an undifferentiated cell (e.g., a stem cell) alters from an undifferentiated state to a differentiated one. Usually, the cell changes to a more specialized type. Differentiation happens multiple times during the development of a multicellular organism as it changes from a simple zygote to a complex system of tissues and cell types. Differentiation continues in adulthood as adult stem cells divide and create fully differentiated daughter cells during tissue repair and during normal cell turnover. Differentiation dramatically changes a cell's size, shape, membrane potential, metabolic activity, and responsiveness to signals. These changes are largely due to highly controlled modifications in gene expression. A specialized type of differentiation, known as terminal differentiation, is of importance in some tissues, for example vertebrate nervous system, striated muscle, epidermis and gut. During terminal differentiation, a precursor cell formerly capable of cell division, permanently leaves the cell cycle, dismantles the cell cycle machinery and often expresses a range of genes characteristic of the cell's final function (e.g., myosin and actin for a muscle cell). Differentiation may continue to occur after terminal differentiation if the capacity and functions of the cell undergo further changes. The methods described herein allow the induction of the differentiation of PSCs into terminally differentiated RPE cells.

In one aspect, the method includes (a) culturing PSCs in conditions that allow growth as an adherent monolayer in a two-dimensional culture system (i.e., not in a three-dimensional culture system); (b) contacting the PSCs with a mixture of agents including one or more of a TGFβ/SMAD2/SMAD3 pathway signaling inhibitor, a BMP/SMAD1/SMAD5/SMAD8 pathway signaling inhibitor, or a FGF/ERK pathway signaling inhibitor; and optionally a TGFβ family protein for about 2-7 days; and (c) subsequently culturing the cells of (b) with the TGFβ family protein for about 4 additional weeks, in the absence of a HIF pathway modulator, in the absence of nicotinamide, and under conditions that are not hypoxic conditions.

In various aspects, the method includes (a) culturing PSCs in conditions that allow growth as an adherent monolayer in a two-dimensional culture system (i.e., not in a three-dimensional culture system); (b) contacting the PSCs with a mixture of agents including about 5 TGFβ/SMAD2/SMAD3 pathway signaling inhibitor, about 0.25 μM BMP/SMAD1/SMAD5/SMAD8 pathway signaling inhibitor, about 0.5-1.5 μM FGF/ERK pathway signaling inhibitor, and about 25 ng/ml Activin A, for about 2-7 days; and (c) subsequently culturing the cells of (b) with about 25 ng/ml Activin A for about 4 additional weeks, in the absence of a HIF pathway modulator, in the absence of nicotinamide, and under conditions that are not hypoxic conditions.

In one aspect, RPE cells differentiation is direct RPE cells differentiation.

By “direct” differentiation, it is meant that the methods described herein allow for obtaining RPE cells from PSCs without intermediately differentiated cells, and without requiring intermediate manipulation of the cells. That is, as opposed to an indirect method, where partially differentiated cells (e.g., multipotent cells, or non-terminally differentiated cells) are obtained in a first process, and are then terminally differentiated into RPE, the method described herein generates RPE cells in a sole process.

Differentiated cells can be characterized based on the differences observed as compared to the undifferentiated cells they are obtained from, including changes in cell's size, shape, membrane potential, metabolic activity, responsiveness to signals, gene expression, etc. In one aspect, RPE characterization include analysis of changes in the expression of genes that form a molecular signature characteristic of RPE cells. Such molecular signature includes for example, the expression of genes selected from PMEL17, MITF, OTX2, BEST1, RPE65, RLBP1, CLDN19, ATP1B1, NC1, ZO1 and/or TYR.

Melanocyte protein PMEL also referred to as pre-melanosome protein is a protein that in humans is encoded by the PMEL gene or PMEL17. PMEL is a 100 kDa type I transmembrane glycoprotein that is expressed primarily in pigment cells of the skin and eye. The transmembrane form of PMEL is modified in the secretory pathway by elaboration of N-linked oligosaccharides and addition and modification of O-linked oligosaccharides. It is then targeted to precursors of the pigment organelle, the melanosome, where it is proteolytically processed to several small fragments. Some of these fragments form non-pathological amyloid that assemble into sheets and form the striated pattern that underlies melanosomal ultrastructure. The expression of the PMEL gene is regulated by the microphthalmia-associated transcription factor (MITF).

Microphthalmia-associated transcription factor also known as class E basic helix-loop-helix protein 32 or bHLHe32 is a protein that in humans is encoded by the MITF gene. MITF is a basic helix-loop-helix leucine zipper transcription factor involved in lineage-specific pathway regulation of many types of cells including melanocytes, osteoclasts, and mast cells. The term “lineage-specific”, since it relates to MITF, means genes or traits that are only found in a certain cell type. Therefore, MITF may be involved in the rewiring of signaling cascades that are specifically required for the survival and physiological function of their normal cell precursors.

Homeobox protein OTX2 is a protein that in humans is encoded by the OTX2 gene. OTX2 is expressed in the brain, ear, nose and eye, and in the case of mutations; it can lead to significant developmental abnormalities and disorders. Mutations in OTX2 can cause eye disorders including anophthalmia and microphthalmia. Apart from anophthalmia and microphthalmia, other abnormalities such as aplasia of the optic nerve, hypoplasia of the optic chiasm and dysplastic optic globes have also been observed. Other defects that occur due to a mutation of the OTX2 gene include pituitary abnormalities and mental retardation. Homeoprotein Otx2 has been identified as a possible molecular ‘messenger’ that is necessary for experience-driven visual plasticity during the critical period. Initially involved in embryonic head formation, Otx2 is re-expressed during the critical period of rats (>P23) and regulates the maturation of parvalbumin-expressing GABAergic interneurons (PV-cells), which control the onset of critical period plasticity.

Bestrophin-1 (Bestl) is a protein that, in humans, is encoded by the BEST1 gene. The bestrophin family of proteins comprises four evolutionary related genes (BEST1, BEST2, BEST3, and BEST4) that code for integral membrane proteins. This family was first identified in humans by linking a BEST1 mutation with Best vitelliform macular dystrophy (BVMD). Mutations in the BEST1 gene have been identified as the primary cause for at least five different degenerative retinal diseases. The bestrophins are an ancient family of structurally conserved proteins that have been identified in nearly every organism studied from bacteria to humans. In humans, they function as calcium-activated anion channels, each of which has a unique tissue distribution throughout the body. Specifically, the BEST1 gene on chromosome 11q13 encodes the Bestrophin-1 protein in humans whose expression is highest in the retina.

Retinal pigment epithelium-specific 65 kDa protein, also known as retinoid isomerohydrolase, is an enzyme of the vertebrate visual cycle that is encoded in humans by the RPE65 gene. RPE65 is expressed in the retinal pigment epithelium (RPE, a layer of epithelial cells that nourish the photoreceptor cells) and is responsible for the conversion of all-trans-retinyl esters to 11-cis-retinol during phototransduction. 11-cis-retinol is then used in visual pigment regeneration in photoreceptor cells. RPE65 belongs to the carotenoid oxygenase family of enzymes. RPE65 is a critical enzyme in the vertebrate visual cycle found in the retinal pigmented epithelium. It is also found in rods and cones. The photoisomerization of 11-cis-retinal to all-trans-retinal initiates the phototransduction pathway through which the brain detects light. All-trans-retinol is not photoactive and therefore must be reconverted to 11-cis-retinal before it can recombine with opsin to form an active visual pigment. RPE65 reverses the photoisomerization by converting an all-trans-retinyl ester to 11-cis-retinol. Most commonly, the ester substrate is retinyl palmitate. The other enzymes of the visual cycle complete the reactions necessary to oxidize and esterify all-trans-retinol to a retinyl ester (RPE65's substrate) and to oxidize 11-cis-retinol to 11-cis-retinal (the required photoactive visual pigment component).

Retinaldehyde-binding protein 1 (RLBP1) also known as cellular retinaldehyde-binding protein (CRALBP) is a 36-kD water-soluble protein that in humans is encoded by the RLBP1 gene. The cellular retinaldehyde-binding protein transports 11-cis-retinal (also known as 11-cis-retinaldehyde) as its physiological ligands. It plays a critical role as an 11-cis-retinal acceptor which facilitates the enzymatic isomerization of all 11-trans-retinal to 11-cis-retinal, in the isomerization of the rod and cones of the visual cycle. Mutations of RLBP1 include several diseases associated with vision. All of these are autosomal recessive including Bothnia dystrophy, retinitis punctata albescens, retinitis pigmentosa, Newfoundland rod-cone dystrophy and fundus albipunctatus. The characteristics of the associated diseases vary with age, severity and rate of progression. These all have similar qualities such as, photoreceptor deterioration and slower dark adaptation, ultimately leading to visual impairment, often leading to complete blindness.

Claudin-19 is a protein that in humans is encoded by the CLDN19 gene. It belongs to the group of claudins. Claudin-19 has been implicated in magnesium transport. Claudins, such as CLDN19, are transmembrane proteins found at tight junctions. Tight junctions form barriers that control the passage of ions and molecules across an epithelial sheet and the movement of proteins and lipids between apical and basolateral domains of epithelial cells

Sodium/potassium-transporting ATPase subunit beta-1 is an enzyme that in humans is encoded by the ATPIBI gene. The protein encoded by this gene belongs to the family of Na+/K+ and H+/K+ ATPases beta chain proteins, and to the subfamily of Na+/K+-ATPases. Na+/K+-ATPase is an integral membrane protein responsible for establishing and maintaining the electrochemical gradients of Na and K ions across the plasma membrane. These gradients are essential for osmoregulation, for sodium-coupled transport of a variety of organic and inorganic molecules, and for electrical excitability of nerve and muscle.

NC1 or non-collagenous 1 (NC1) is a protein domain from COL4A1. COL4A1 belongs to the type IV collagen family and contains three domains: a short N-terminal domain, a long triple-helical 7S domain at its center, and a non-collagenous 1 (NC1) domain at its C-terminal. The triple-helical domain contains interrupted G-X-Y repeats, which is suspected to allow flexibility of the domain. The NC1 domain is composed of two trimeric caps, each containing two alpha 1 fragments and one alpha 2 fragment, that form a sixfold propeller arranged around an axial tunnel. The interaction between these two caps occurs along a large planar interface and is stabilized by a covalent cross-link between the alpha 1 and alpha 2 chains across the two caps.

Zonula occludens-1 ZO-1, also known as tight junction protein-1 is a 220-kD peripheral membrane protein that is encoded by the TJP1 gene in humans. It belongs to the family of zonula occludens proteins (ZO-1, ZO-2, and ZO-3), which are tight junction-associated proteins and of which, ZO-1 is the first to be cloned. It has a role as a scaffold protein which cross-links and anchors Tight Junction (TJ) strand proteins, which are fibril-like structures within the lipid bilayer, to the actin cytoskeleton.

Tyrosinase is an oxidase that is the rate-limiting enzyme for controlling the production of melanin. The enzyme is mainly involved in two distinct reactions of melanin synthesis otherwise known as the Raper Mason pathway. Firstly, the hydroxylation of a monophenol and secondly, the conversion of an o-diphenol to the corresponding o-quinone. o-Quinone undergoes several reactions to eventually form melanin. Tyrosinase is a copper-containing enzyme present in plant and animal tissues that catalyzes the production of melanin and other pigments from tyrosine by oxidation. It is found inside melanosomes which are synthesized in the skin melanocytes. In humans, the tyrosinase enzyme is encoded by the TYR gene. A mutation in the tyrosinase gene resulting in impaired tyrosinase production leads to type I oculocutaneous albinism, a hereditary disorder that affects one in every 20,000 people. If uncontrolled during the synthesis of melanin, Tyr activity results in increased melanin synthesis. Decreasing tyrosinase activity has been targeted for the improvement or prevention of conditions related to the hyperpigmentation of the skin, such as melasma and age spots.

In various aspect, differentiated RPE cells have an increased expression of PMEL17, MITF, OTX2, BEST1, RPE65, RLBP1 and/or TYR as compared to hPSCs.

In many aspects, the differentiated RPE cells do not express any pluripotent stem cell markers (i.e., they are not pluripotent after the differentiation process, and are terminally differentiated). Stem cell markers include genes that are expressed by stem cells, and include but are not limited to ECAT11, OCT4, NANOG, SOX2, mir302HT and LIN28.

In one aspect, the differentiated RPE cells do not express ECAT11, OCT4, NANOG, SOX2, mir302HT nor LIN28.

In an additional embodiment, the invention provides a method of treating macular degeneration in a subject including administering to the subject differentiated retinal pigment epithelium (RPE) cells, wherein differentiated RPE cells are obtained by one of the methods described herein.

Dysfunction of the RPE is associated with various retinal degenerative diseases. As used herein, the term “retinal degenerative disease” or “degenerative retinopathy” is meant to refer to any retinopathy which consists in the deterioration of the retina caused by the progressive death of its cells. There are several reasons for retinal degeneration, including artery or vein occlusion, diabetic retinopathy, R.L.F./R.O.P. (retrolental fibroplasia/retinopathy of prematurity), or disease (usually hereditary). These may present in many different ways such as impaired vision, night blindness, retinal detachment, light sensitivity, tunnel vision, and loss of peripheral vision to total loss of vision. Of the retinal degenerative diseases retinitis pigmentosa (RP) is a very important example. Non-limiting example of degenerative retinopathy that are associated with RPE deterioration include macular degeneration, such as age-related macular degeneration (AMD), retinitis pigmentosa, diabetic retinopathy, and Gardner syndrome (characterized by FAP (familial adenomatous polyps), osseous and soft tissue tumors, retinal pigment epithelium hypertrophy and impacted teeth).

The methods described herein are particularly directed to the treatment of age-related macular degeneration (AMD) in a subject, comprising administering to the subject, the RPE cells obtained by the methods described herein.

Age-related macular degeneration or AMD is a disease that affects a person's central vision. It can result in severe loss of central vision, but people rarely go blind from it. AMD is the most common cause of severe loss of eyesight among people 50 and older. Only the center of vision is affected with this disease. AMD affects the central vision, and with it, the ability to see fine details. In AMD, a part of the retina called the macula is damaged. In advanced stages, people lose their ability to drive, to see faces, and to read smaller print. In its early stages, AMD may have no signs or symptoms, so people may not suspect they have it. The two primary types of age-related macular degeneration have different causes. Dry AMD, the most common type (about 80% of those with AMD have the dry form) is of unknown exact cause, although both genetic and environmental factors are thought to play a role. This happens as the light-sensitive cells in the macula slowly break down, generally one eye at a time. The loss of vision in this condition is usually slow and gradual. It is believed that the age-related damage of an important support membrane under the retina contributes to dry age-related macular degeneration. Wet AMD is the less common type, and usually leads to more severe vision loss in patients than dry AMD. It is the most common cause of severe loss of vision. Wet AMD happens when abnormal blood vessels start to grow beneath the retina. They leak fluid and blood—hence the name wet AMD—and can create a large blind spot in the center of the visual field. The methods described herein are useful for the treatment of both wet and dry AMD in a subject.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures). The terms “therapeutically effective amount”, “effective dose,” “therapeutically effective dose”, “effective amount,” and the like refer to that amount of the RPE cells that will elicit the biological or medical response of a tissue, system, animal or human that is being sought by the researcher, veterinarian, medical doctor or other clinician. Generally, the response is either amelioration of symptoms in a patient or a desired biological outcome (e.g.,treating AMD). Such amount should be sufficient to treat AMD in the subject. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intraorbital, intravitreal, subretinal, and any other ocular administrations, as well infusion.

In one aspect, administering differentiated RPE cells comprises injecting RPE cells in situ.

Currently, there is no treatment for dry age-related macular degeneration, though vision rehabilitation programs and low-vision devices can be used to build visual skills, develop new ways to perform daily living activities and adjust to living with age-related macular degeneration. The main treatment for wet AMD is the injection anti-VEGF agents, as high level of VEGF in the eye is linked to the formation of the abnormal blood vessels that cause much of the damage in wet AMD. Anti-VEGF agents are used to combat the disease process and reduce the damaging effects of these leaky abnormal blood vessels. They are also able to effectively stabilize vision in many patients.

In some aspects administration of the RPE cells described herein can be in combination with one or more additional therapeutic agents. The phrases “combination therapy”, “combined with” and the like refer to the use of more than one medication or treatment simultaneously to increase the response. The RPE cells of the present invention might for example be used in combination with other drugs or treatment in use to treat AMD. Specifically, the administration of RPE cells to a subject can be in combination with anti-VEGF agents. Such therapies can be administered prior to, simultaneously with, or following administration of the RPE cells of the present invention.

In one aspect, administering differentiated RPE cells increases photoreceptor function and/or survival.

“Photoreceptor” or “photoreceptor cell”, as used herein refers to a specialized type of neuroepithelial cell found in the retina that is capable of visual phototransduction. The great biological importance of photoreceptors is that they convert light (visible electromagnetic radiation) into signals that can stimulate biological processes. Specifically, photoreceptor proteins in the cell absorb photons, triggering a change in the cell's membrane potential. There are three known types of photoreceptor cells in mammalian eyes: rods, cones, and intrinsically photosensitive retinal ganglion cells. The two classic photoreceptor cells are rods and cones, each contributing information used by the visual system to form a representation of the visual world, sight. Rods primarily contribute to night-time vision (scotopic conditions) whereas cones primarily contribute to day-time vision (photopic conditions), but the chemical process in each that supports phototransduction is similar. Intrinsically photosensitive retinal ganglion cells are thought not to contribute to sight directly but have a role in the entrainment of the circadian rhythm and pupillary reflex.

There are major functional differences between the rods and cones. Rods are extremely sensitive and can be triggered by a single photon. At very low light levels, visual experience is based solely on the rod signal. Cones require significantly brighter light (that is, a larger number of photons) to produce a signal. In humans, there are three different types of cone cell, distinguished by their pattern of response to light of different wavelengths. The three types of cone cell respond (roughly) to light of short, medium, and long wavelengths, so they may respectively be referred to as S-cones, M-cones, and L-cones.

The human retina contains about 120 million rod cells, and 6 million cone cells. The number and ratio of rods to cones varies among species, dependent on whether an animal is primarily diurnal or nocturnal. In the human visual system, in addition to the photosensitive rods and cones, there are about 2.4 million to 3 million ganglion cells, with 1 to 2% of them being photosensitive. The axons of ganglion cells form the two optic nerves. Photoreceptor cells are typically arranged in an irregular but approximately hexagonal grid, known as the retinal mosaic.

Rod and cone photoreceptors are found on the outermost layer of the retina; they both have the same basic structure. Closest to the visual field (and farthest from the brain) is the axon terminal, which releases a neurotransmitter called glutamate to bipolar cells. Farther back is the cell body, which contains the cell's organelles. Farther back still is the inner segment, a specialized part of the cell full of mitochondria. The chief function of the inner segment is to provide ATP (energy) for the sodium-potassium pump. Finally, closest to the brain (and farthest from the field of view) is the outer segment, the part of the photoreceptor that absorbs light. Outer segments are actually modified cilia that contain disks filled with opsin, the molecule that absorbs photons, as well as voltage-gated sodium channels.

The membranous photoreceptor protein opsin contains a pigment molecule called retinal. In rod cells, these together are called rhodopsin. In cone cells, there are different types of opsins that combine with retinal to form pigments called photopsins. Three different classes of photopsins in the cones react to different ranges of light frequency, a differentiation that allows the visual system to calculate color. The function of the photoreceptor cell is to convert the light information of the photon into a form of information communicable to the nervous system and readily usable to the organism: This conversion is called signal transduction.

By “increasing photoreceptor function and/or survival”, it is meant that the administration of RPE cells increase function and/or survival of any type of photoreceptors, including rods, S-cones, M-cones, L-cones, and intrinsically photosensitive retinal ganglion cells.

In some aspects, increasing photoreceptor function comprises increasing renewal of photoreceptor outer segment, increasing phagocytosis involving MERTK and/or increasing, restoring and/or creating cell/cell tight junctions.

In various aspects, restoring and/or creating cell/cell tight junctions improves or restores brain-eye barrier.

The “blood-ocular barrier” or “brain-eye barrier” is a barrier created by endothelium of capillaries of the retina and iris, ciliary epithelium and retinal pigment epithelium. It is a physical barrier between the local blood vessels and most parts of the eye itself and prevents many substances including drugs from traveling across it. Inflammation can break down this barrier allowing drugs and large molecules to penetrate into the eye. As the inflammation subsides, this barrier usually returns. It consists of a blood-aqueous barrier including the ciliary epithelium and capillaries of the iris, and a blood-retinal barrier including non-fenestrated capillaries of the retinal circulation and tight-junctions between retinal epithelial cells. Blood-aqueous barrier is formed by nonpigmented ciliary epithelial cells of the ciliary body and endothelial cells of blood vessels in the iris. Blood-retinal barrier prevents the passage of large molecules from choriocapillaris into the retina and if formed by endothelium of retinal vessels and epithelium of retinal pigment.

In another aspect, PSCs are hPSCs. In some aspects, hPSCs are autologous hPSCs or allogenic hPSCs.

hPSCs can be obtained from the subject in need of a treatment, i.e., autologous hPSCs. Autologous hPSCs have the advantage of eliminating virtually all chance of graft versus host disease rejection, as there is no risk of incompatibility and therefore risk of any issues associated with incompatibility. When hPSCs cannot be obtained from the subject itself, hPSCs can be obtained from an allogenic, non-autologous donor, that has been matched for histocompatibility with the subject. Allogenic hPSCs offer the advantages of being prepared/stored in advance for use as soon as necessary. However, there remains the risk associate with the lack of histocompatibility between the donor and the recipient, and the risk of reject of the cells (e.g., acute or chronic graft versus host disease).

In a further embodiment, the invention provides a kit comprising: (a) a neuroectodermal induction cocktail including: a TGFβ/SMAD2/SMAD3 pathway signaling inhibitor, a BMP/SMAD1/SMAD5/SMAD8 pathway signaling inhibitor, and/or an FGF/ERK pathway signaling inhibitor; (b) a TGFβ family protein; and (c) instructions to induce pluripotent stem cells (PSCs) differentiation into retinal pigment epithelium (RPE) cells.

In one aspect, the kit further includes a laminin-coated surface.

Presented below are examples discussing methods of inducing the differentiation of PSCs into RPE cells, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1 Design of Retinal Pigmented Epithelium Cells Differentiation Protocol

In a stepwise iterative developmental process, a new combination of factors promoting superior RPE differentiation at high efficiency has been identified using a simple and GMP-compatible workflow. The basic principle is a combination of a neuroectodermal induction cocktail of small molecules together with Activin A treatment in a defined temporal sequence (see FIG. 1 for an illustrative example of a method of the invention). In brief, a combination of two or three pathway inhibitors comprising an FGF/ERK signaling inhibitor (e.g., PD0325901, “PD”, or similar), a TGFβ/SMAD2/3 signaling inhibitor (e.g., SB431542, “SB”), and a BMP/SMAD1/5/8 inhibitor (e.g., Dorsomorphin, “DM”, or similar) was used to prime the iPSCs for the neuroectodermal lineage. Inhibiting both SMAD pathways with or without additional inhibition of FGF/ERK signaling for prolonged periods of time is known to give rise to neuronal fates (not RPE), and RPE are not neuronal cells. Treatment of iPS cells with the neural induction cocktail, even for shorter periods of time, does not markedly induce RPE over background. Activin A is known to have a positive signaling effect on RPE differentiation in vivo. However, Activin A is a key ligand promoting self-renewal of hPSCs not differentiation.

However, it was discovered that their use for limited on hPSCs primed hPSCs for differentiation into RPE cells, when combined with Activin A.

As illustrated in FIG. 1, in an illustrative method, it is the combination Activin A treatment and SMAD/FGF pathway inhibition that gives rise to highly efficient RPE induction (i.e., high purity, and in shorter period) under adherent culture conditions.

### Example 2 Material and Methods

Maintenance of hiPSCs

Before initiating differentiation, hiPSCs were maintained on iMatrix-511 in iPS Brew medium and passaged using EDTA splitting as for routine culture.

Cells were then grown to 90-100% confluency before start, and on 4x iMatrix-511, that is, on 6-wells coated with 12 μl instead of 3 μl iMatrix-511 per 6-well. Full confluency at start yielded better differentiation outcomes than starting from semiconfluent cultures. However, the experiment can be performed with semi confluent starting conditions. Enhancing laminin coating in initial differentiation phase appeared to have positive effects on morphology and pigmentation, but perhaps more importantly in later stages, after replating RPE cells. 12 μl per 6-well of coating seemed optimal for RPE maintenance.

The week before differentiation, a desired number of 6-wells were coated with 125 μl iMatrix-511 per 6-well in 2 ml PBS each for 1 h at 37° C. or overnight at 2-8° C. hiPSCs were split using ˜4 min EDTA digestion time or, for better controlling cell density and homogeneity, TrypLE Select with 10 μM Rocki supplementation in the new culture wells, aiming at 90-100% confluency. In the meantime, cells were fed as appropriate, including a 6-8 ml per well media change.

Differentiation of hiPSCs

Day 0: overall undifferentiated and tight hiPSC morphology was confirmed to be at close to 100% confluency. Otherwise, the cells were fed once more with 4 ml prewarmed iPS Brew medium and instead differentiation was initiate the next day. KSR medium (4 ml needed per 6-well), PD0325901 (PD)/SB431542 (SB)/dorsomorphin (DM) aliquots, as well as Activin A were prepared and/or prewarmed. After thawing and thoroughly mixing all reagents, 18 μM PD (1:1000 from 1 mM stock), 59 μM SB10 (1:2000 from 10 mM stock), 0.2511 μM DM (1:2000 from 0.5 mM stock) and 2512 ng/ml Activin A13 (1:400 from 10 μg/ml stock) were added to 4 ml KSR medium per confluent hiPSC culture well, after vigorously agitating culture plates and completely soaking off iPS Brew medium.

Days 1 and 2: media was prepared as above and old medium replaced on culture wells by 4 ml fresh differentiation medium per well, following vigorous agitation and complete media aspiration.

Day 3: KSR medium with Activin A only was prepared and old medium was replaced on culture wells by 4 ml fresh ActA-only differentiation medium per well, following vigorous agitation and complete media aspiration.

Day 4: fresh KSR+ActA differentiation medium was prepared and feed with 8 ml per well.

Second week: cells were fed daily with 4 ml per 6-well and 8 ml over the weekend.

Third and fourth weeks: no more small molecules were applied during the third and fourth weeks of differentiation. Only Activin A was added to basal media (either KSR or B27 medium) Activin A was added daily to prewarmed basal media, and old medium replaced on cells (1:100 thoroughly mixed).

Day 29 (week 5): newly coated dishes were prepared by coating 12-well (5 μl) or 6-well format (12 μl iM) with 4 x iMatrix-511 in in PBS for 1 h at 37° C. KSR medium was prepared and prewarmed for splitting procedure. Differentiated cells were washed with PBS once (4 ml) or twice (2×2 ml), then 1 ml prewarmed TrypLE Se-lec was added. Cells were dissociated using a 1 ml pipette by pipetting up and down several times and results were checked under the microscope. Cell suspension was transferred to 15 ml tube and centrifuged at 300 g for 2 min. A portion of the cells was transferred into a 1.5 ml tube for downstream FACS and/or RT-qPCR analysis. Cells were resuspended in an appropriate amount of KSR medium and cell titer was determined using a Neubauer chamber. 400,000 cells per cm² were plated out in 6-well (with 4 ml KSR medium with or without Activin A24) or 12-well format (2 ml medium).

Day 31: cells were fed with KSR medium (6-well format: 4 ml, 12-well format: 2 ml).

Day 33: cells were feed with KSR medium (6-well format: 8 ml, 12-well format: 4 ml).

Week 6: week 5 procedure was repeated. Depending on the state of the culture (morphology, results of in-process controls), the cells were either passaged again (for further cleanup or expansion), or left sitting for maturation in KSR medium without Activin A.

As required for specific characterization assays, RPE cells in Px1 or beyond (xn=passage after initial differentiation) were replated onto transwells. In brief, 24-well transwells with 0.33 cm² surface area were coated with 0.5 μl iMatrix-511 per well. Depending on the RPE maturation stage (Px1 vs. Px2 or higher, e.g., with prolonged incubation over several weeks), cells were replated with 1x or 10x TrypLE29. After PBS wash, cells were digested for 10 min and result checked under microscope followed by gentle pipetting with a 1 ml pipette, followed by re-check under microscope. If vast majority of cells were single cells, digestion was stopped by adding 3 volumes of KSR medium, and cells were transferred to 15 ml tube and centrifuged for 2 min at 300 g. Otherwise incubation time was prolonged, and result checked as above. Pelleted cells were resuspended in appropriate volume of KSR medium, cell titer was determined using a Neubauer chamber, and 100,000 cells were plate out per well—total inner volume: 0.5 ml, total outer volume: 1 ml. medium was replaced twice per week until timepoint of analysis.

Analysis by RT-qPCR

Samples to be analyzed by RT-qPCR were cells directly lysed from culture wells using RA1 buffer from the RNA isolation kit. For instance, culture medium was soaked off after vigorous agitation, then 600 μl buffer RA1 was added to culture well and sample was lyzed and homogenized by quickly pipetting up and down with a 1 ml pipette. 350 μl was transferred to 1.5 ml tube or directly onto a filtration column and kit instructions were followed. Alternatively, a representative fraction of cells (e.g., 25% of a 6-well) was scraped out from a running culture using a sharp plastic scraper, followed by pipetting the floating clumps off into a 1.5 ml tube. The tube was briefly spined e.g., on a mini centrifuge and completely remove supernatant (ideally no liquid remaining on top of cell pellet). Then cells were quickly resuspended in 350 μl buffer RA1. The sample was immediately homogenized after adding buffer RA1. As a third option, cells were harvested for additional purposes such as replating or parallel FACS analysis. In this case, cells were harvested by generating a cell suspension, and a suitable fraction of it was pipetted into a 1.5 ml tube for RNA isolation. The cells were centrifuged on a mini or benchtop centrifuge and used as described above. RNA was isolated according to the manufacturers' instructions using an elution volume of 40 μl. RNA concentration was measured on NanoDrop monitoring A260/280 and A260/230 ratios. RNA were kept cold at all times. cDNA synthesis reaction was assembled in a PCR strip and incubated for 1 h at 42° C. in a PCR machine:
18.5 μl diluted RNA, same amount for all samples, e.g., μg or 500 ng
5 μl 5 x RT buffer (as part of master mix)
0.5 μl dNTP mix (as part of master mix)
0.5 μl oligo-dT primer (as part of master mix)
0.5 μl M-MLV reverse transcriptase (as part of master mix)

cDNA reactions were diluted by adding 150 μl water each. qPCRs were assembled according to experiment-specific template using gene-specific primers:
10 μl SYBR Green mix
3 μl gene-specific primer working mix
7 μl diluted cDNA

qPCR was run according to corresponding GMP SOPs, and analyze data based on experiment-specific template.

Analysis by FACS

Aliquot of FACS buffer was prepared or thawed to assemble primary and secondary antibody solutions (200 μl per sample and antibody):
MITF1 antibody 1:150>A11001 2nd antibody 1:750-1:1000
BEST1 antibody 1:150>A11001 2nd antibody 1:750-1:1000
PMEL17 antibody 1:50>A21206 2nd antibody 1:750-1:1000
ZO1 antibody 1:250>A21206 2nd antibody 1:750-1:1000

Secondary antibodies-only samples served as negative controls. Cells were harvested as for splitting RPE yielding a single-cell suspension. Following cell counting, defined numbers of cells were transferred into required number of 1.5 ml tubes (range 0.5-1 Mio cells per staining). Cells were centrifuged at 400 g for 1 min, supernatant was discarded, pellet was washed with 180-200 μl PBS, pellet was generated again, and supernatant was discarded. Pellets were resuspended in 400 μl 2% formaldehyde/PBS each, quickly pipetted up and down several times, incubated at RT for ˜10 min. Tubes were centrifuged at 400 g for 1 min, supernatant was completely soaked off, cells were resuspended in 180-200 μl FACS buffer, tubes were let stand for 1-2 min to block, centrifuged again, and supernatant was discarded. Cells were then resuspended in primary antibody solution, incubated for 15 min at RT, tube was flicked at least once during incubation, centrifuged as before, and supernatant soaked off. Cells were washed as before using 180-200 μl FACS buffer. Cells were resuspended in appropriate secondary antibody solution, incubated for 15 min at RT, tube was flicked at least once during incubation, centrifuged as before, and supernatant was soaked off. Cells were washed and centrifuged with 180-200 μl FACS buffer as before, each cell pellet was resuspended in 300 μl PBS and analyzed on flow cytometer using suitable template. Percent of positive cells was based on hierarchical gating on (i) cells, (ii) single cells, and (iii) encircled positive cloud in fluorescence intensity vs. forward scatter plots.

### Example 3 Characterization of Retinal Pigmented Epithelium Cells Differentiated from Human Pluripotent Stem Cellss

Human induced pluripotent stem cells (hiPSCs) were differentiated into RPE following the protocol described in Example 2 and in the illustrative method represented in FIG. 1.

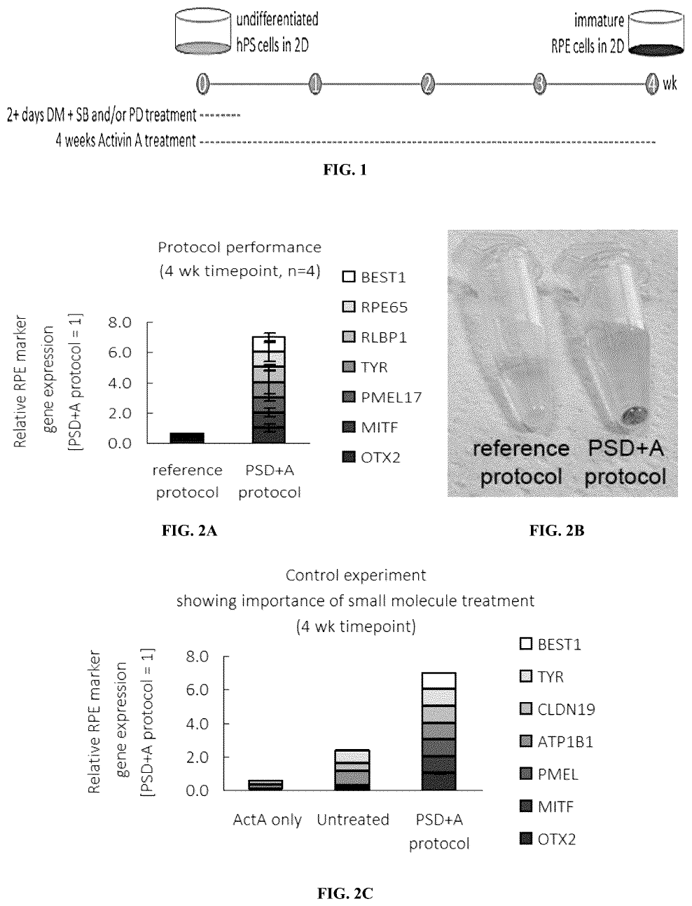

The protocol described herein, using PD0325901 (PD, or P)/SB431542 (SB, or S)/dorsomorphin (DM, or D) aliquots, as well as Activin A (A), is referred as the PSD+A protocol. The PSD+A protocol was initially compared the procedure described by Maruotti et al., (2015). As illustrated in FIGS. 2A-2C, the PSD+A protocol yielded, at a 4-week timepoint, RPE cells that expressed higher levels of expression of BEST1, RPE65, RLBP1, TYR, PMEL17, MITF and OTX2 as measured by RTqPCR and as compared to the REF cells obtained using the reference protocol (see FIG. 2A). As shown in FIG. 2B, this corresponded to RPE cells having a pigmentation phenotype significantly different from the cell obtained by the reference protocol. FIG. 2C further illustrates that treatment with A alone was not sufficient to induce the RPE phenotype, and that PSD+A was the optimal protocol, showing the importance of the small molecules treatment.

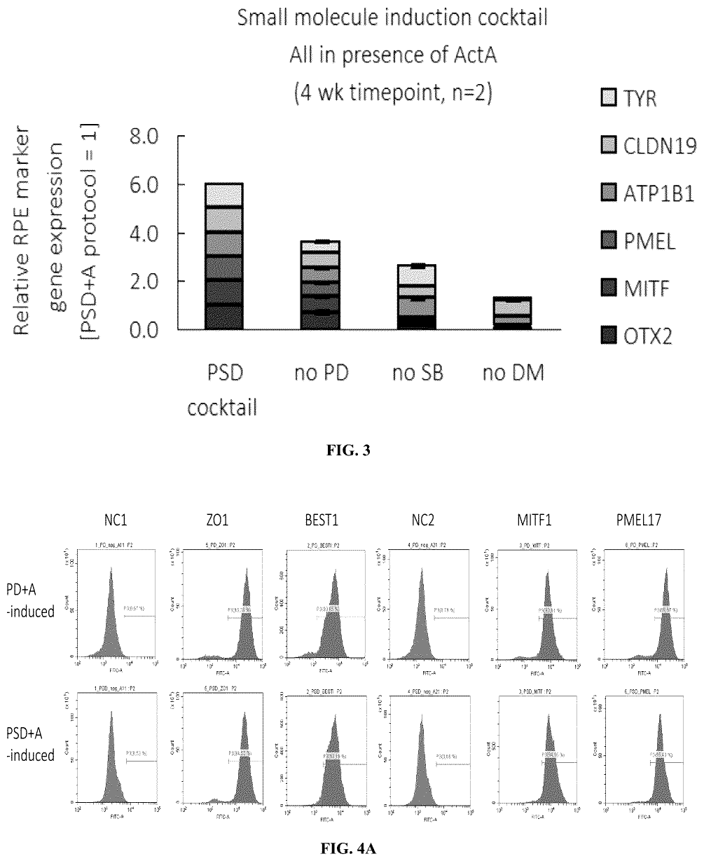

As illustrated in FIG. 3, RPE gene expression was quantified by RTqPCR at 4 weeks, following an initial induction with PSD, SD (no PD), PD (no SB) or PS (no DM). DM withdrawal showed the most severe decrease in RPE-specific markers expression as compared to the standard PSD protocol, and may therefore be considered essential, while PD and SB withdrawal may be tolerated in the long term.

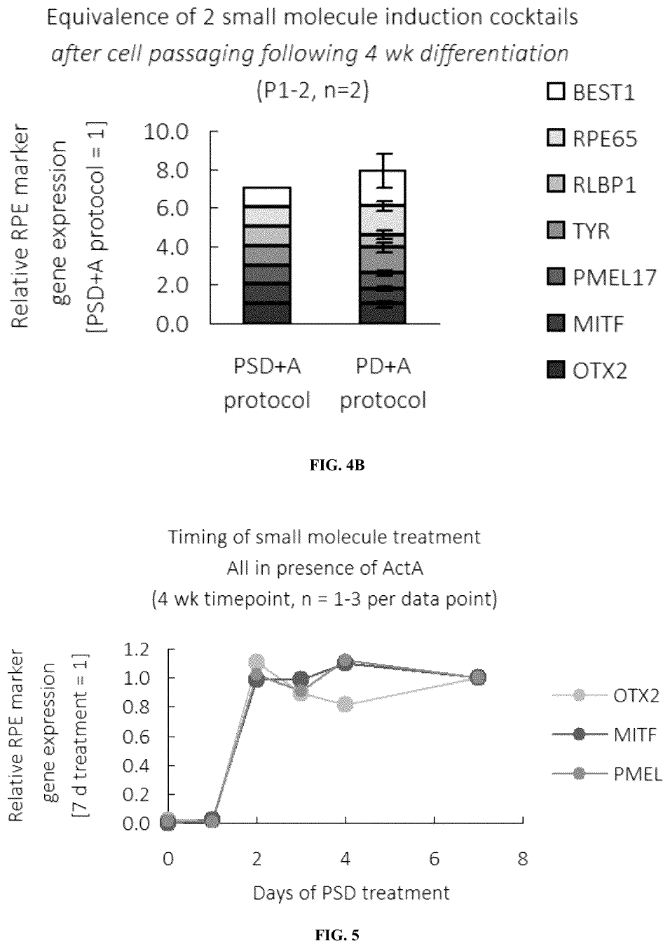

As shown in FIGS. 4A-4B, long-term equivalence of PSD+A and PD+A induction following replating of cells treated for 4 weeks was assessed. iPS cells were induced with a 2-factor small molecule cocktail (PD) or with a 3-factor cocktail (PSD), and RPE-specific markers expression were evaluated after the initial 4 weeks of differentiation. FIG. 4A shows the levels of protein expression as measured by flow cytometry, and FIG. 4B shows the levels of gene expression as measured by RTqPCR. The results showed that although induction with the tree pathway inhibitors is preferred, 2 factors-induced RPE cells presented equivalent RPE-specific markers expression and may therefore be equivalent.

The timing of the three-pathway inhibition was evaluated to assess how long the cells needed to be contacted with the three small molecules to induce RPE cell differentiation. As shown in FIG. 5, the RTqPCR analysis showed that 1 day of treatment or a complete withdrawal (Activin A alone) was insufficient for inducing RPE cell fate, but that 2 days or more (at least up to 7 days) was optimal, with little differences observed between the different exposure times.

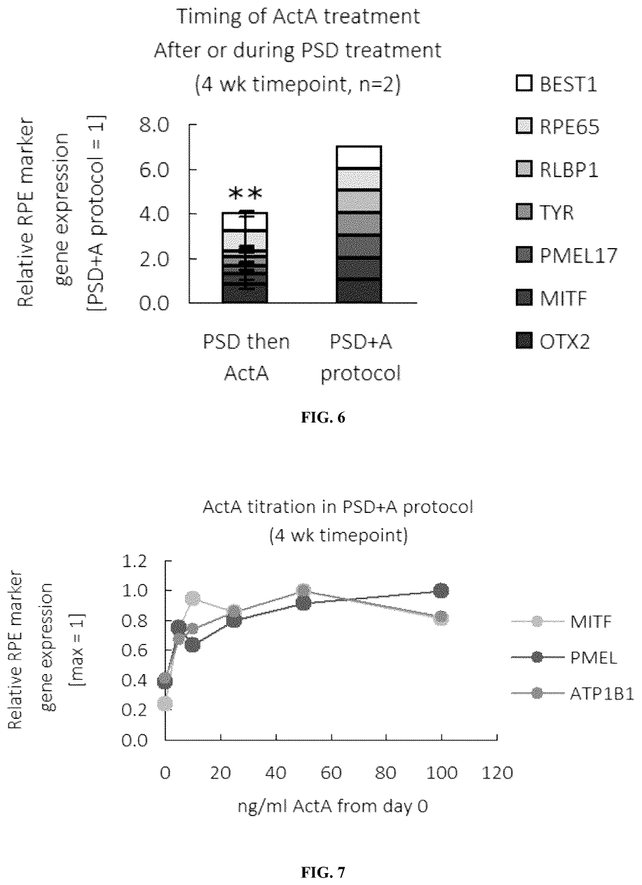

It was thus evaluated when the treatment with Activin A should be initiated for optimal RPE induction, and which concentration should be used. As illustrated in FIG. 6, variation from the standard protocol in the form of initiating Activin A treatment after signaling inhibition yielded reduced RPE gene expression, suggesting the importance to treat the cells with the small molecule inhibitors and with Activin A simultaneously to obtain optimal RPE cell induction. As illustrated in FIG. 7, titration of Activin A revealed that concentration below 20 ng/ml or complete withdrawal of Activin A yielded inferior RPE induction, but not a complete absence of RPE induction.

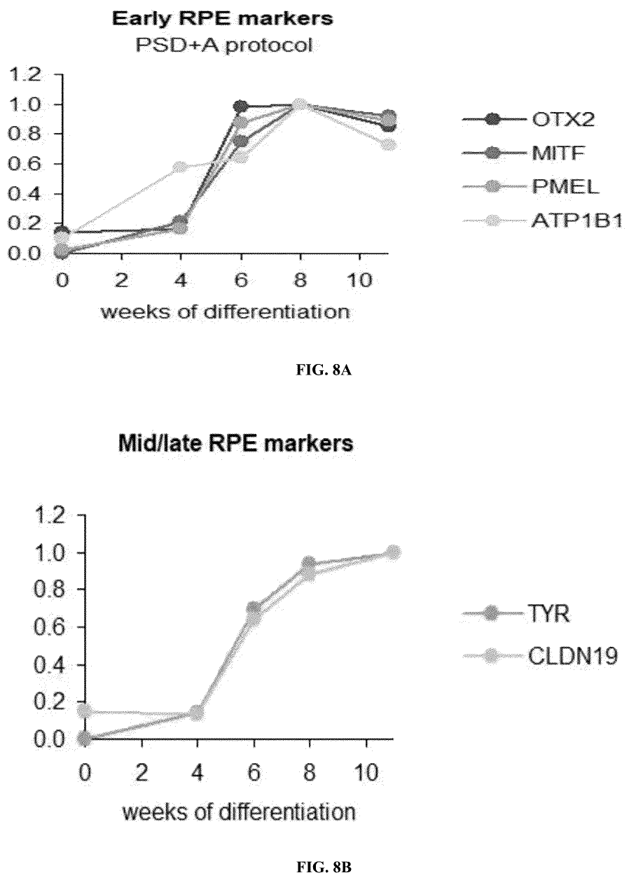

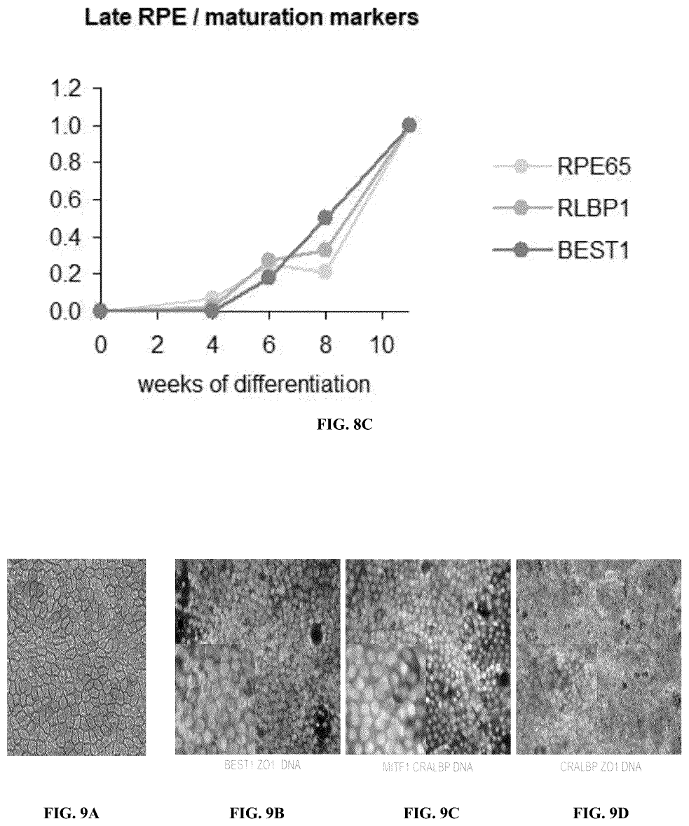

A time course of RPE markers was assessed, to evaluate the level of expression of early, mid/late, late and maturation markers up to 10 weeks after RPE induction. As shown in FIGS. 8A-8C, PSD+A induced early (FIG. 8A)/mid/late (FIG. 8B), late/maturation (FIG. 8C) RPE markers at different stages of differentiation following a several-days induction.

RPE cells were also characterized by immunofluorescence and using light microscopy. As illustrated in FIG. 9A, RPE cells shown a typical pigmented cobblestone morphology in phase contrast light and were found to expressed RPE markers BEST1 (FIG. 9B), ZO1 (FIGS. 9B and 9D), MITF1 (FIG. 9C) and CRALBP (FIGS. 9C and 9D).

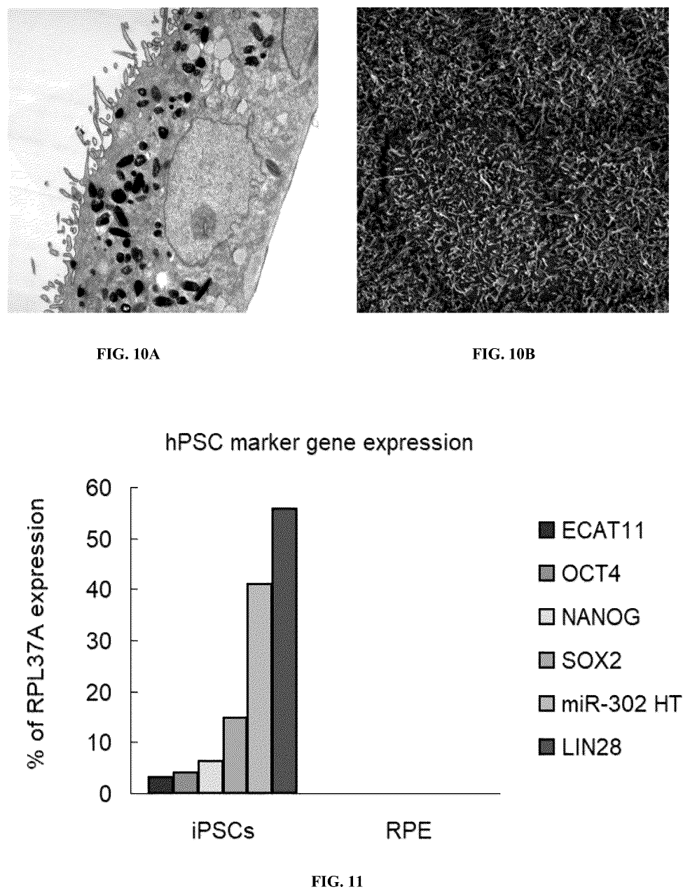

RPE cells were further analyzed using electron microscopy, as shown in FIGS. 10A-10B. Transmission (FIG. 10A) and scanning (FIG. 10B) electron microscopy analysis of RPE cells generated with the PSD+A protocol showed typical RPE features such as epithelial polarity with nuclei located at the basal side, and melanosomes as well as microvilli toward the apical side and on the apical cell surface, respectively.

Finally, the terminal differentiation of the cells was assessed, by evaluating if the cells had any remaining expression of any stem cell markers. As shown in FIG. 11, RPE cells obtained by the PSD+A protocol were assessed for ECAT11, OCT4, NANOG, SOX2, mir-302 HT and LIN28 expression. The levels of expression were compared to those in iPSCs. As illustrated in FIG. 11, RPE cells did not present any stem cell marker expression.

### References

Almedawar, S., Vafia, K., Schreiter, S., Neumann, K., Khattak, S., Kurth, T., Ader, M., Karl, M. O., Tsang, S. H., and Tanaka, E. M. (2020). MERTK-Dependent Ensheathment of Photoreceptor Outer Segments by Human Pluripotent Stem Cell-Derived Retinal Pigment Epithelium. Stem Cell Reports 14, 374-389.
Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nat Biotechnol 27, 275-280.
da Cruz, L., Fynes, K., Georgiadis, O., Kerby, J., Luo, Y. H., Ahmado, A., Vernon, A., Daniels, J. T., Nommiste, B., Hasan, S. M., et al. (2018). Phase 1 clinical study of an embryonic stem cell-derived retinal pigment epithelium patch in age-related macular degeneration. Nat Biotechnol 36, 328-337.
Fuhrmann, S., Levine, E. M., and Reh, T. A. (2000). Extraocular mesenchyme patterns the optic vesicle during early eye development in the embryonic chick. Development 127, 4599-4609.
Greber, B., Coulon, P., Zhang, M., Moritz, S., Frank, S., Muller-Molina, A. J., Arauzo-Bravo, M. J., Han, D. W., Pape, H. C., and Scholer, H. R. (2011). FGF signalling inhibits neural induction in human embryonic stem cells. EMBO J 30, 4874-4884.
Greber, B., Lehrach, H., and Adjaye, J. (2008). Control of early fate decisions in human ES cells by distinct states of TGFbeta pathway activity. Stem Cells Dev 17, 1065-1077.
Greber, B., Wu, G., Bernemann, C., Joo, J. Y., Han, D. W., Ko, K., Tapia, N., Sabour, D., Sterneckert, J., Tesar, P., et al. (2010). Conserved and divergent roles of FGF signaling in mouse epiblast stem cells and human embryonic stem cells. Cell Stem Cell 6, 215-226.
Idelson, M., Alper, R., Obolensky, A., Ben-Shushan, E., Hemo, I., Yachimovich-Cohen, N., Khaner, H., Smith, Y., Wiser, O., Gropp, M., et al. (2009). Directed differentiation of human embryonic stem cells into functional retinal pigment epithelium cells. Cell Stem Cell 5, 396-408.
Mandai, M., Kurimoto, Y., and Takahashi, M. (2017). Autologous Induced Stem-Cell-Derived Retinal Cells for Macular Degeneration. N Engl J Med 377, 792-793.

Maruotti, J., Sripathi, S. R., Bharti, K., Fuller, J., Wahlin, K. J., Ranganathan, V., Sluch, V. M., Berlinicke, C. A., Davis, J., Kim, C., et al. (2015). Small-molecule-directed, efficient generation of retinal pigment epithelium from human pluripotent stem cells. Proc Natl Acad Sci USA 112, 10950-10955.
Osakada, F., Jin, Z. B., Hirami, Y., Ikeda, H., Danjyo, T., Watanabe, K., Sasai, Y., and Takahashi, M. (2009). In vitro differentiation of retinal cells from human pluripotent stem cells by small-molecule induction. J Cell Sci 122, 3169-3179.
Plaza Reyes, A., Petrus-Reurer, S., Antonsson, L., Stenfelt, S., Bartuma, H., Panula, S., Mader, T., Douagi, I., Andre, H., Hovatta, O., et al. (2016). Xeno-Free and Defined Human Embryonic Stem Cell-Derived Retinal Pigment Epithelial Cells Functionally Integrate in a Large-Eyed Preclinical Model. Stem Cell Reports 6, 9-17.
Rao, J., and Greber, B. (2017). Concise Review: Signaling Control of Early Fate Decisions Around the Human Pluripotent Stem Cell State. Stem Cells 35, 277-283.
Sharma, R., Khristov, V., Rising, A., Jha, B. S., Dejene, R., Hotaling, N., Li, Y., Stoddard, J., Stankewicz, C., Wan, Q., et al. (2019). Clinical-grade stem cell-derived retinal pigment epithelium patch rescues retinal degeneration in rodents and pigs. Sci Transl Med 11.
Xu, R. H., Sampsell-Barron, T. L., Gu, F., Root, S., Peck, R. M., Pan, G., Yu, J., Antosiewicz-Bourget, J., Tian, S., Stewart, R., et al. (2008). NANOG is a direct target of TGFbeta/activin-mediated SMAD signaling in human ESCs. Cell Stem Cell 3, 196-206.
Zahabi, A., Shahbazi, E., Ahmadieh, H., Hassani, S. N., Totonchi, M., Taei, A., Masoudi, N., Ebrahimi, M., Aghdami, N., Seifinej ad, A., et al. (2012). A new efficient protocol for directed differentiation of retinal pigmented epithelial cells from normal and retinal disease induced pluripotent stem cells. Stem Cells Dev 21, 2262-2272.
Zhu, Y., Carido, M., Meinhardt, A., Kurth, T., Karl, M. O., Ader, M., and Tanaka, E. M. (2013). Three-dimensional neuroepithelial culture from human embryonic stem cells and its use for quantitative conversion to retinal pigment epithelium. PLoS One 8, e54552.

Although the invention has been described with reference to the above examples, it will be understood that modifications and variations are encompassed within the spirit and scope of the invention. Accordingly, the invention is limited only by the following claims.

What is claimed is:

1. A method of generating retinal pigment epithelium (RPE) cells comprising:
(a) contacting a culture of pluripotent stem cells (PSCs) with a mixture of agents comprising:
(i) one or more of a transforming growth factor TGFβ (TGFβ)/SMAD2/SMAD3 pathway signaling inhibitor, a bone morphogenetic protein (BMP)/SMAD1/SMAD5/SMAD8 pathway signaling inhibitor, or a fibroblast growth factor (FGF)/ERK pathway signaling inhibitor; and
(ii) optionally, a TGFβ family protein; and
(b) subsequently culturing the cells of (a) with the TGFβ family protein in the absence of the mixture of the agents of (a)(i),
thereby generating RPE cells.

2. The method of claim 1, wherein the culture of PSCs is an adherent monolayer of cells.

3. The method of claim 2, wherein the monolayer of cells is grown in a two-dimensional culture system.

4. The method of claim 1, wherein the TGFβ/SMAD2/SMAD3 pathway signaling inhibitor is selected from the group consisting of SB431542, LY3200882, TP0427736 HCl, RepSox, SB525334, GW788388, BIBF-0775, SD-208, galunisertib, vactosertib, A-83-01, LY2109761, SB505124, LY364947 and LDN-212854.

5. The method of claim 1, wherein the BMP/SMAD1/SMAD5/SMAD8 pathway signaling inhibitor is selected from the group consisting of dorsomorphin, PPM1A and LDN-193189.

6. The method of claim 1, wherein the FGF/ERK pathway signaling inhibitor is selected from the group consisting of PD0325901, PD173074, SU431542, PD161570, PD98059, PD184352, PD198306 and PD334581.

7. The method of claim 1, wherein the TGFβ family protein is selected from the group consisting of Activin A, TGFβ1, TGFβ2 and TGFβ3.

8. The method of claim 1, wherein the mixture of (a) comprises about 0.1-10 μM TGFβ/SMAD2/SMAD3 pathway signaling inhibitor, about 0.1-1 μM BMP/SMAD1/SMAD5/SMAD8 pathway signaling inhibitor, and/or about 0.5-5 μM FGF/ERK pathway signaling inhibitor; and at least about 0.1 ng/ml of a TGFβ family protein.

9. The method of claim 1, wherein the mixture comprises about 1-5 μM SB431542, about 0.25 μM dorsomorphin, about 0.5-1.5 μM PD0325901, and about 25 ng/ml Activin A.

10. The method of claim 1, wherein contacting PSCs with the TGFβ family protein in (b) comprises contacting the PSCs with at least about 0.1 ng/ml TGFβ family protein.

11. The method of claim 1, wherein contacting PSCs with the TGFβ family protein in (b) comprises contacting the PSCs with about 25 ng/ml Activin A.

12. The method of claim 1, wherein contacting PSCs with the mixture of (a) is for about 2-7 days.

13. The method of claim 1, wherein contacting PSCs with the TGFβ family protein in (b) is for about 4 weeks.

14. The method of claim 3, wherein the monolayer is cultured on a surface comprising a laminin coating.

15. The method of claim 1, wherein the PSCs are human PSCs (hPSCs).

16. The method of claim 15, wherein the hPSCs are human induced pluripotent stem cells (hiPSCs) or human embryonic stems cells (hESCs).

17. The method of claim 1, wherein contacting PSCs with the mixture is in the absence of a hypoxia inducible factor (HIF) pathway modulator.

18. The method of claim 1, wherein contacting PSCs with the mixture is in the absence of nicotinamide.

19. The method of claim 1, wherein the PSCs are cultured under conditions that are not hypoxic conditions.

20. The method of claim 1, wherein the PSCs are cultured in a system that is not in a three-dimensional culture system.

21. A method of inducing retinal pigment epithelium (RPE) cell differentiation from pluripotent stem cells (PSCs) comprising:
(a) culturing PSCs in conditions that allow growth as an adherent monolayer in a two-dimensional culture system;
(b) contacting the PSCs with a mixture of agents comprising one or more of a transforming growth factor TGFβ (TGFβ)/SMAD2/SMAD3 pathway signaling inhibitor, a bone morphogenetic protein (BMP)/SMAD1/SMAD5/SMAD8 pathway signaling inhibitor or a fibroblast growth factor (FGF)/ERK pathway signaling inhibitor, and optionally a TGFβ family protein for about 2-7 days; and
(c) subsequently culturing the cells of (b) with the TGFβ family protein for about 4 additional weeks,
thereby inducing RPE cell differentiation from PSCs.

22. The method of claim 21, wherein RPE cell differentiation is direct RPE cell differentiation.

23. The method of claim 21, wherein differentiated RPE cells have an increased expression of PMEL17, MITF, OTX2, BEST1, RPE65, RLBP1, CLDN19, ATP1B1, NC1, ZO1 and/or TYR as compared to hPSCs.

24. The method of claim 21, wherein differentiated RPE cells do not express ECAT11, OCT4, NANOG, SOX2, mir302HT nor LIN28.

25. The method of claim 21, wherein the PSCs are human PSCs (hPSCs).

26. A method of treating macular degeneration in a subject comprising administering to the subject differentiated retinal pigment epithelium (RPE) cells,
wherein differentiated RPE cells are obtained by the method of claim 1.

27. The method of claim 26, wherein administering differentiated RPE cells increases photoreceptor function and/or survival.

28. The method of claim 27, wherein increasing photoreceptor function comprises increasing renewal of photoreceptor outer segment, increasing phagocytosis involving MERTK and/or increasing, restoring and/or creating cell/cell tight junctions.

29. The method of claim 28, wherein restoring and/or creating cell/cell tight junctions improves or restores brain-eye barrier.

30. The method of claim 26, wherein administering differentiated RPE cells comprises injecting RPE cells in situ.

31. The method of claim 26, wherein the PSCs are human PSCs (hPSCs).

32. The method of claim 31, wherein the hPSCs are autologous hPSCs or allogenic hPSCs.

33. A kit comprising:
(a) a neuroectodermal induction cocktail comprising: a transforming growth factor β (TGFβ)/SMAD2/SMAD3 pathway signaling inhibitor, a bone morphogenetic protein (BMP)/SMAD1/SMAD5/SMAD8 pathway signaling inhibitor, and/or a fibroblast growth factor (FGF)/ERK pathway signaling inhibitor;
(b) a TGFβ family protein; and
(c) instructions for inducing pluripotent stem cells (PSCs) differentiation into retinal pigment epithelium (RPE) cells.

34. The kit of claim 33, further comprising a laminin-coated surface.
